(19) United States
(12) Patent Application Publication
(10) Pub. No.: US 2009/0036844 A1
(43) Pub. Date: Feb. 5, 2009

(54) Reservoir with liquidly applied seal

(75) Inventors: Charlotte Juel Fristrup, Lyngby (DK); Lasse Hansen, Brondby Strand (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/628,293

(22) PCT Filed: May 31, 2005
(86) PCT No.: PCT/DK05/00359

§ 371 Date: Jan. 2, 2008

Related U.S. Application Data
(60) Provisional application No. 60/580,602, filed on Jun. 17, 2004.

(30) Foreign Application Priority Data
Jun. 7, 2004 (DK) PA 2004 00886

(51) Int. Cl. A61M 31/00; A61J 1/10; B05D 3/00; A61J 1/14

(52) U.S. Cl. 604/288.04; 604/408; 604/415; 427/2.3

(57) ABSTRACT

The present invention provides a fluid reservoir comprising a first wall portion formed from a needle-penetrable material, the first wall portion comprising a seal surface. The reservoir further comprises a seal member formed from a needle-penetratable self-sealing material, wherein the seal member has been applied on the seal surface in a liquid state. In this way a liquid reservoir having a self-sealing needle-penetrable access means which can be manufactured in a cost-effective manner is provided.

The present invention generally relates to a reservoir for the storage of fluids, the reservoir comprising access means allowing the interior of the container to be accessed by a fluid conducting means such as a penetrating hollow needle member. The reservoir may be designed to contain in particular medical liquid products such as drugs, drug solutions, infusion solutions, parenteral solutions, dialysis solutions, perfusion solutions, chemical and alimentary liquids, human blood and its fractions, and the like. The container may also be used for other purposes, e.g. calibration liquids for analytical equipment.

### BACKGROUND OF THE INVENTION

In the disclosure of the present invention reference is mostly made to liquids in the form of medical liquids as outlined above, however, this is only an exemplary use of the present invention.

Medical liquids are often supplied in reservoirs (also termed containers in the alternative) which can be accessed by means of a hollow needle, the needle typically penetrating a dedicated connection element (or access means) of the reservoir to provide a fluid communication with the interior of the container. The needle access may be used either for withdrawing liquids from the reservoir or for supplying a liquid to the reservoir. For example, when preparing the fluids which are to be administrated to the body of a patient from a given reservoir, it is common that medically effective substances are supplied to a pre-sealed reservoir which is filled with a transport fluid, usually in the form of sodium chloride solution or a glucose solution, the diluted drug then being given to a patient intravenously via an intravenous (IV) infusion set. For this type of use, the reservoir may be provided with a single connection means adapted to be used for both purposes, or the reservoir may comprise two connection means adapted for their respective purposes, e.g. for a larger infusion set outlet needle and a smaller drug injecting needle. The connection element may be adapted to be opened manually to provide an opening, through which a needle subsequently can be inserted, or the pointed needle can be used for penetrating the connection element, which may be of the self-sealing type, e.g. the connection element will seal the container after the needle has been withdrawn.

In case the reservoir is of glass, the connection element will be a separate element which is mounted to the glass reservoir by special means, however, for plastic reservoir the connection element will typically be formed integrally with the reservoir. One of the most widely used type of plastic containers for medical use is in the form of a flexible infusion bag comprising at a lower portion thereof one or more needle-penetrable connection elements. Such bags are typically formed from flexible foil sheets which are joined to form an internal space. Depending on the actual construction of the bag, the connection element(s) may be arranged either on a surface portion of a foil sheet or may be arranged corresponding to an edge portion of the container. For the latter type, the connection element is typically positioned and held in place between two foil sheets connected to each other by welding. For both of the above two arrangements, the self-sealing element is normally carried by a tubular member connected to the bag in either of the above two ways. For example, U.S. Pat. No. 4,362,158 shows an infusion bag in which a tubular nozzle member is connected to an infusion bag corresponding to a free surface thereof, a self-sealing rubber seal member being mounted on the nozzle. DE 42 34 957 discloses a flexible medical container comprising a seal member arranged on an inside surface of the bag.

For some liquids, e.g. certain types of drugs, it is desirable if the elastomeric material from which the seal member normally is manufactured, does not come in contact with drug. To solve this problem, European patent application EP 0 364 783 describes a medicament bottle having an external sealing element held in place by a separate cap member attached to the outer surface of the bottle, and FR 2 752 410 discloses a needle-penetrable fluid container (IV bag) where a septum member has been mounted on an outer surface by means of welding.

The above reservoir are relatively large, typically comprising 100-1.000 ml of liquid, however, containers which can be accessed by a penetrating needle member is also used for much smaller volumes. For example, certain calibration solutions for calibrating analytical equipment are supplied in small bag-like containers containing a few ml. When properly designed, such small containers may also be used for drug purposes.

### DISCLOSURE OF THE INVENTION

Having regard to the above-identified known reservoirs, it is an object of the present invention to provide a liquid reservoir having a self-sealing needle-penetrable access means which can be manufactured in a cost-effective manner. It is a further object of the invention to provide a reservoir which is convenient and safe in use and allows a varity of applications. It is a yet further object of the invention to provide a reservoir which can be used with a varity of liquids. It is a yet further object of the invention to provide a reservoir device in which a fluid access device is adapted to be arranged in fluid communication with the interior of a container. Further objects and advantages will become apparent from the disclosure of the invention and the description of the exemplary embodiments.

In the disclosure of the present invention, embodiments and aspects will be described which will address one or more of the above objects or which will address objects apparent from the below disclosure as well as from the description of exemplary embodiments.

More specifically, the present invention provides in a first aspect a reservoir containing a fluid, comprising a first wall portion formed from a needle-penetrable material, the first wall portion comprising an outer seal surface. The reservoir further comprises a seal member formed from a needle-penetrable self-sealing material, wherein the seal member has been applied on the seal surface in a liquid state (including a paste). As will be apparent to the skilled person, the method of application will provide a finished product in which the method of application will be structurally recognisable. The material for the seal surface may be located solely corresponding to the placement of the seal member, or it may cover a portion or the entire exterior surface of the container. In the alternative, the reservoir may be adapted to contain a fluid and filled at a later stage.

Although the term “self-sealing” will be clear to the skilled person, it should be noted that this is not to be regarded as an absolute term for a given seal member but that it will depend upon the intended use for a given container. For example, a given seal member will be designed to be self-sealing in connection with needles of a given range of gauges (i.e. diameters) and with a given design for the pointed distal end. Thus, a relatively thin seal member adapted to be used with a correspondingly thin needle may not be self-sealing when penetrated by a larger needle. Further, if the container is pressurized above the intended internal pressure, a punctuated seal member may leak.

By the term “adapted to contain” is defined that a reservoir in accordance with embodiments of the invention may be provided in an empty state for subsequent filling, or that it may be filled during manufacture of the reservoir.

The term “seal member” is defined as an amount of material having the defined functionality at the defined location, i.e. it may have any given form and configuration and may be either solid (i.e. a cured material) or it may remain more or less liquid, e.g. gel-like. Suitable materials for the seal member of the present invention can be found in the group of materials used traditionally as adhesives. To properly seal between the reservoir and the seal member, the seal member should have good adhesion to the seal surface, which adhesion may be improved by corona treatment or other treatment of the seal surface.

In contrast to a traditional self-sealing septum which has to be welded to or otherwise mounted to the reservoir before it is filled, the liquid seal can be applied after both filling and sterilization of the reservoir have taken place, this allowing for e.g. a more flexible production and a greater freedom in the choice of materials. Further, the liquid seal can be used to help attach the reservoir to a desired structure.

To control the application of the liquid seal material during manufacture, a cavity-forming structure is advantageously provided forming a cavity in which the seal surface is at least partially arranged. Alternatively the seal surface may be arranged in the vicinity of the cavity. In this way the cavity can be filled with the liquid seal material which will then be located at the desired location. The cavity may be designed to be filled with an exact amount of material or it may have openings allowing it to be filled until “overrun” of seal material. In case the seal material remains liquid to a certain degree in the finished product, the cavity will ensure that the seal member is not dislocated e.g. during transportation, storage or use.

The cavity-forming structure may be formed integrally with the reservoir or it may be attached thereto as a separate structure. For example, the cavity may be established between a structure to which the reservoir is to be mounted and a portion of the reservoir, which cavity is then filled with the seal material after mounting. The cavity-forming structure may be permanently attached to the reservoir or it may be utilized as a mould for forming a seal of a given configuration, the mould being removed after curing. The seal material may also be applied freely on the reservoir surface using a nozzle or by any other suitable means e.g. printing or spraying techniques.

The reservoir may also be arranged relative to a mounting structure with a gap formed between the mounting structure and the seal surface, the seal member being arranged at least partially in the gap bridging a portion of the mounting structure and the seal surface. In this way a curable seal material may be used to attach and hold the reservoir in place relative to the mounting structure.

If the fluid (liquid or gas) to be contained in the reservoir does not require specific properties for the surrounding walls (e.g. in respect of evaporation, leakage or chemical inertness), the first wall portion may be made from a single layer of material or the reservoir in general may be formed from a single material. If the fluid requires specific properties for the reservoir, the reservoir may be provided with an outer surface generally formed from a first material and an inner surface generally formed from a second material. The wall portion providing the seal surface may be of a special construction (e.g. allowing needle-penetration) with the rest of the reservoir wall having different properties.

The reservoir may be a relative rigid container (e.g. a blown bottle) or a more flexible reservoir as a traditional IV bag. For the latter, the container may comprise at least first and second flexible foil (or film) members sealed together to form at least one enclosed cavity for containing the liquid. The foil members may be provided as two or more separate members or as a single member which is folded upon itself. The foil members may be composite laminates of continuous layers including an outer layer and an inner layer. This would allow the outer surface to be optimized for connection of the septum whereas the inner surface may be optimized in respect of reservoir properties in respect of the fluid to be contained. Any laminate referred to in the present application may be a traditional laminate, a co-extrudate or an extrusion-laminate. The walls defining the reservoir may be formed from a single material or single type of laminate, or different materials or laminates may be used for different wall portions of the reservoir.

The foil members may comprise an intermediate layer, the inner layer being formed from a weldable material, which would allow the foil members to be sealed together at least partially by welding corresponding to the peripheries of the bag. In case the outer layer solely provides the mounting surface for the septum, the “intermediate” layer may provide the outer layer for a portion of the reservoir. To allow a contained liquid to be viewed through the reservoir wall, at least a portion thereof may be formed by a transparent or translucent material.

As the seal member in accordance with an aspect of the invention is attached directly to an outer wall portion of the reservoir without the need of using additional elements or means (e.g. adhesives) to secure the seal member, the latter can be configured to provide additional properties in addition to the sealing properties. For example, a portion of the seal member or the entire seal member may be configured to flex together with the portion of the reservoir to which it is mounted, this allowing the seal member to be bend either during manufacture or during use. To allow this flexibility the seal member may be in the form of a relatively thin member or may comprise a relatively thin portion. Further, when the seal member is arranged “naked” on the reservoir, it may be accessed from a wide range of angles.

Although the seal member may be mounted alone, it may be desirable to provide additional mounting means allowing access means such a tubing to be connected to the reservoir, e.g. as described above with respect to the known IV bags. For example, a tubular connector protruding from the surface of the reservoir may be attached around the seal member, e.g. circumferentially. In this way the two components can be attached independently of each other without having the seal member to be mounted to the connector, although it may be convenient to attach the two components to the reservoir simultaneously during manufacture.

In a further aspect of the invention a reservoir device is provided comprising a reservoir of the type as disclosed above, where the seal member serves as a reservoir outlet. The device further comprises a fluid communication structure such as a pointed hollow needle structure having an inlet and an outlet, the inlet being adapted to be arranged in fluid communication with the reservoir via the reservoir outlet, wherein the fluid communication structure and the reservoir are moveable relative to each other from an initial position in which there is no fluid communication therebetween and a connected position in which the fluid communication structure inlet is arranged in fluid communication with the reservoir through the reservoir outlet.

In an exemplary embodiment, the device further comprises a delivery assembly adapted to move a fluid (e.g. a drug) contained in the reservoir through the outlet of the reservoir. The delivery assembly may be adapted to force or suck the drug from the reservoir. In the latter case the delivery assembly may be in the form of a pump (e.g. a membrane pump) having an inlet and an outlet, the inlet being adapted to be arranged in fluid communication with the reservoir through the fluid communication structure.

The reservoir and the delivery assembly may be arranged moveable relative to each other, or they may both be fixed relative to a housing, the fluid communication being provided by a moveable fluid communication structure which advantageously is formed as part of the delivery assembly. The seal member may be used to provide a connection between the reservoir and the delivery assembly, e.g. the seal member may provide a bridge between the reservoir and the delivery assembly, the seal member also providing a seal for an opening in the delivery assembly through which the fluid communication structure can be moved.

The seal member may be mounted to a housing with the reservoir being attached to the housing primarily corresponding to the seal member and optionally the neighbouring portions of the reservoir. In this way a secure fixation between the reservoir and the housing is provided without having to interfere with the flexibility of the reservoir, e.g. substantially the entire flexible reservoir apart from the seal member may be arranged free to move relative to the housing, this allowing the reservoir to be emptied to a high degree.

In a further aspect a method for manufacturing a reservoir containing or adapted to contain a fluid is provided, the method comprising the steps of providing a reservoir comprising a first wall portion formed from a needle-penetrable material, the first wall portion comprising an outer seal surface, and applying a liquid material to the seal surface, the liquid material being adapted to form a needle-penetrable self-sealing seal member. In exemplary embodiments the reservoir is provided in combination with a cavity-forming structure forming a cavity in which the seal surface is at least partially arranged, the cavity being arranged to receive at least a portion of the applied liquid material. The method may comprise the further step of curing the liquid material or allowing the liquid material to cure if the applied material is selfcuring, thereby forming the needle-penetrable self-sealing seal member. In exemplary embodiments the reservoir is sealed and contains an amount of a fluid. The seal member and the materials forming the reservoir may be selected in accordance with the above described embodiments. For example, the wall portion providing the first mounting surface may be manufactured from or comprise polyethylene (PE), polypropylene (PP), oriented polypropylene (OPP), amorphous polyester (PET), oriented amorphous polyester (OPET), polyamide (PA), oriented polyamide (OPA) or PCTFE. The seal member may be manufactured from e.g. 1- or 2-component adhesives which e.g. may be curable by exposure to moisture or radiation, e.g. UV radiation.

As used herein, the term “drug” is meant to encompass any drug-containing flowable medicine capable of being passed through a delivery means such as a hollow needle in a controlled manner, such as a liquid, solution, gel or fine suspension. Representative drugs indude pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject.

Herein the term “insulin” refers to insulin from any species such as porcine insulin, bovine insulin, and human insulin and salts thereof such as zinc salts, and protamin salts as well as active derivatives of insulin, and insulin analogues. The term “active derivatives of insulin”, is what a skilled art worker generally considers derivatives, vide general textbooks, for exampie, insulin having a substituent not present in the parent insulin molecule. The term “insulin analogues” refers to insulin wherein one or more of the amino acid residues have been exchanged with another amino acid residue and/or from which one or more amino acid residue has been deleted and/or from which one or more amino acid residue has been added with the proviso that said insulin analogue has a sufficient insulin activity.

The material(s) used to form the reservoir or reservoir devices of the present invention may be selected in accordance with the intended use. Thus it may be required that the material(s) fulfil(s) specified functional requirements such as physical properties for the material after sterilization, chemical requirements for the material after sterilization, and cleanliness. Correspondingly, the material may be sterilizeable using e.g. gamma irradiation, electron beam, steam, or ethylene oxide. The material(s) may further be selected in accordance with one or more of the following requirements: 1) the material must be transparent, 2) the material must provide a good barrier against water evaporation; 3) the material must provide a good barrier against gasses (for example, oxygen and carbon dioxide); 4) the material must provide a good barrier against preservatives (for example, phenol and meta-cresol); 5) the material must provide a good barrier against odors (for example preservatives); 6) the material must be resistant against environmental stress cracking (for example, oils, perfumes); 7) the material must be resistant against flex-crack; 8) the material must have good sealing properties (for example, by welding); 9) the material must not delaminate after sterilization, during processing or storage; 10) the material must not relax significantly during storage and use, 11) the material must not emit substances to the drug which can affect the health and safety of the patient (leachables); 12) the material must have a very low level of extractables; and 13) the material must be compatible with a contained drug formulation. It may further be relevant that the material(s) fulfil(s) certain health and safety requirements, preferably most of or all the requirements mentioned in 1) European Pharmacopoeia (Ph. Eur.) 2002, 4^{th} edition; 2) The United States Pharmacopeia (USP) 25; 3) Japanese Pharmacopeia (JP) XIV; 4) EEC Directive 90/128+amendments “Relating to plastics materials and articles intended to come into contact with foodstuffs”; 5) Code of federal regulations (CFR) Title 21 Food and Drugs, part 170-190; 6) 111/9090/90 EN. Plastic Primary Packaging Materials. Note for Guidance; and 7) Guidance for Industry. Container Closure Systems for Packaging Human Drugs and Biologics, Chemistry, Manufacturing, and Controls Documentation. FDA, May 1999.

In respect of laminates the following definitions are used: Co-extrusion covers a process where two or more polymer materials are melded in two or more extruders and co-extruded together through a flat nozzle or systems of flat nozzles and cooled to form the co-extruded foil. Extrusion-lamination covers a process where a feedstock in form of a foil of one material is coated through a flat nozzle or systems of flat nozzles from one or more extruders with one layer or more layers of melted material or materials and then cooled to form the extrusionlamination foil. “Traditional” lamination covers a process, where two feed stocks of foil materials are joined together by adding a proper adhesive to one foil, followed by addition of the second foil forming the laminated foil. A tie layer is a layer which is placed between two polymer layers with the object of securing that the two layers are joined together.

### BRIEF DESCRIPTION OF THE DRAWINGS

In the following the invention will be further described with references to the drawings, wherein

FIG. 1 shows a side view of an infusion bag,

FIG. 2 shows a cross-sectional view along line II-II of FIG. 1,

FIG. 3 shows a cross-sectional view of an alternative connecting means for an infusion bag,

FIG. 4 shows a perspective exploded view of a drug delivery device,

FIGS. 5A and 5B show a pump unit and reservoir connection,

FIG. 6A shows an exploded view of a pump assembly,

FIG. 6B shows a cross-sectional view of the pump assembly of FIG. 6A, and

FIGS. 6C and 6D show partial cross-sectional views of the pump assembly of FIG. 6A.

In the figures like structures are identified primarily by like reference numerals.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

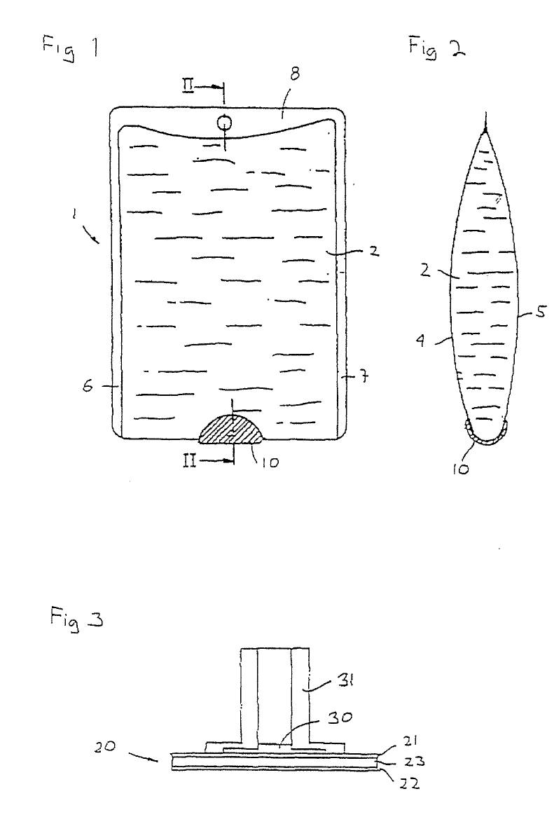

FIG. 1 shows a side view of an infusion bag which is generally denoted with 1. The infusion bag 1 exhibits an internal space 2 which is surrounded by walls 3 of a preferably flexible material, the material being penetrable by a pointed hypodermic needle. In the shown embodiment a piece of foil is folded upon itself corresponding to a lower edge 7 of the bag, thereby forming a front wall 4 connected to a back wall 5 along two opposite side edges 6, 7 and an upper edge 8. The connection can be accomplished by means of welding or, alternatively, gluing. In this manner, the internal space is formed between the joined edges and the lower folded edge. However, the invention is not limited to a special design of an infusion bag, but it can be manufactured in any way which is well-known to the skilled person. The foil may comprise one or more layers, e.g. for a saline solution the foil may be a single layer of polyethylene (PE).

The bag further comprises a seal member formed from a needle-penetrable self-sealing material, the seal member being formed from an elastomeric material allowing it to be applied directly onto the outer surface of the bag in a liquid state. In the shown embodiment the cured seal member is arranged at the lower rounded edge of the bag, however, it may be arranged at any desirable location just as the bag may be provided with additional connection means (not shown), e.g. a tubular member allowing an infusion set to be connected and held in place. The liquid seal material may be applied using any suitable technique, e.g. by printing, spraying or by using a mould held against the bag.

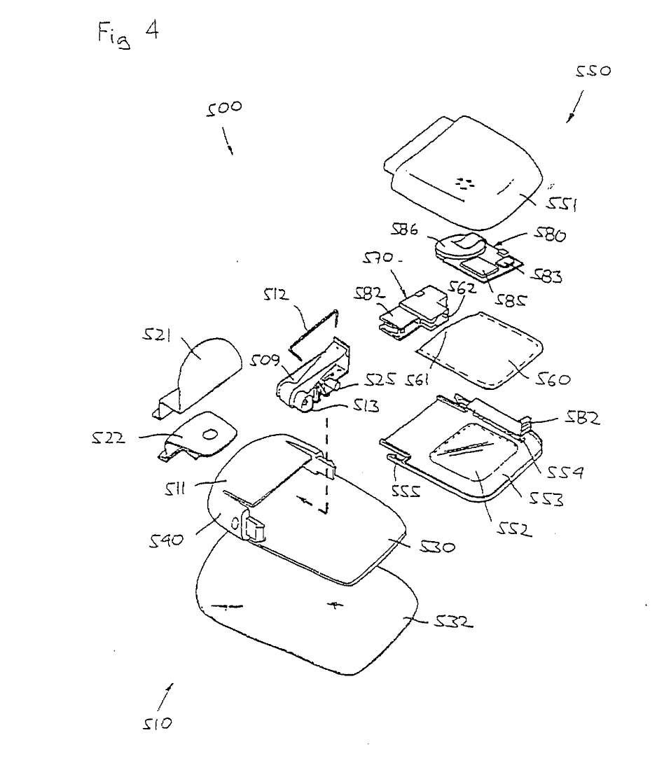

FIG. 3 shows a wall portion 20 of a further bag to which a seal member 30 formed from a needle-penetrable self-sealing material has been applied. The wall is formed from a laminate comprising an outer weldable layer 21 allowing the seal member to be welded thereto, an intermediate layer 23 providing a barrier layer for an enclosed fluid, and an inner weldable layer 22 allowing the bag to be formed by welding. In addition to the seal a tubular member 31 is attached circumferentially in respect to the seal, preferably by welding or adhesives (e.g. using the same material forming the seal member), thereby providing a connection means for connecting e.g. an infusion set to the bag. The seal and the tube may be attached in the same process or independently of each other. When the tube (or another cavity forming member) is mounted first it may provide a cavity for the subsequently applied liquid seal material. The outer layer of the bag may be dispensed with. In FIG. 4 an embodiment of a drug delivery device is shown, the device comprising a flexible reservoir allowing one or more aspects of the present invention to be implemented. More specifically, FIG. 4 shows a drug delivery device 500 comprising a transcutaneous device unit 510 having a housing portion 511 and a base portion 530 attached to a flexible adhesive patch member 532 having a lower adhesive surface providing a mounting surface per se. The transcutaneous device unit 510 comprises a transcutaneous device in the form of a hollow infusion needle and will thus in the following be termed a needle unit, however, the needle may be replaced with any desirable transcutaneous device suitable for delivery of a fluid drug, e.g. a soft cannula in combination with an insertion needle. Within the housing portion 511 a needle actuation unit 509 is arranged, the needle actuation unit comprising a hollow infusion needle 512 pivotally arranged relatively to the base plate.

The inlet portion of the needle is arranged corresponding to the pivoting axis, the inlet portion being protected by a cylindrical member 513 protecting the user against accidental needle pricks. The needle actuation unit 509 is adapted for moving the needle between a retracted and extended state, and retraction means (not shown) is provided for moving the needle between the extended and a retracted position. The actuation and retraction means are actuated by gripable first and second strip members 521, 522 connected to the respective means through slot-formed openings in the housing. The second strip is further connected to the patch 532. The needle unit further comprises male 540 and female (not shown) coupling members adapted to cooperate with corresponding female and male 555 coupling members on the reservoir unit, as well as an actuator 525 for establishing fluid communication between the pump unit and the reservoir (see below).

The reservoir unit 550 comprises a housing, formed from upper and lower housing portions 551, 553, in which a reservoir and expelling assembly are arranged, the expelling assembly comprising a pump assembly 570 and a controller assembly 580 therefore. The lower housing portion comprises two windows 552, 554 allowing the user to inspect the content of the reservoir respectively a reservoir indicator (see below). The reservoir 560 is in the form of prefilled, flexible and collapsible pouch formed from a flexible foil folded corresponding to one edge of the reservoir and sealed along the remaining three edges. In the shown state the needle-penetrable seal member has not yet been applied to the rounded, folded edge. The pump assembly which in the shown embodiment is in the form of a membrane pump comprises a pump actuating member in the form of a coil actuator 582 operatively connected to thereto, and a mounting means in the form of a slot 562 allowing the rounded portion 561 of the reservoir to be arranged there within, in which position the liquid seal material can be applied, this providing both the seal member and allows the reservoir to be mounted and fixed relative to the inlet of the pump.

The controller assembly comprises a microprocessor 583 for controlling, among other, the pump actuation, a signal generator 585 for generating an audible and/or tactile signal, and an energy source 586. A reservoir indicator 582 for indicating to the user an amount of drug left in the reservoir is coupled to the controller assembly. The indicator may be in the form of an electrochemical strip of the type used in e.g. batteries.

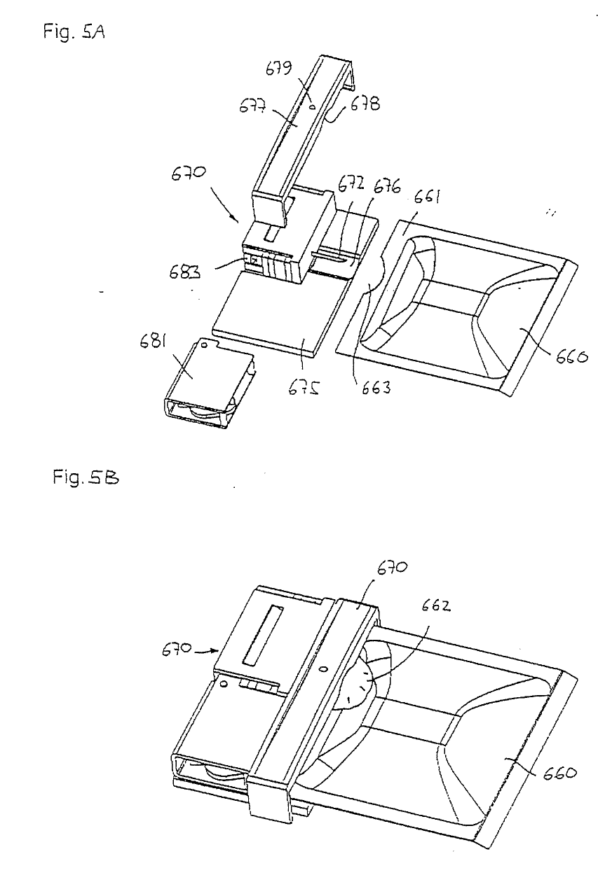

With reference to FIGS. 5A and 5B an alternative configuration for the reservoir mounting means is shown. The pump unit 670 comprises a base plate 675 with a first groove portion 676 and a clamp member 677 with a second groove portion 678. The base plate and the clamp member are adapted to engage opposed surfaces of the reservoir edge portion 661 with the two groove portions forming a cavity around a part of the edge portion into which a liquid seal material 662 can be introduced through an opening 679 in the clamp member when the reservoir has been locked in place as shown in FIG. 5B, thereby placing and securing the seal member relative to the pump. To allow the seal material to properly form a single seal around the edge portion 661 of the reservoir, a cut-out portion 663 is provided in the edge. The groove portions may be provided with additional gripping elements (e.g. protrusions, not shown) preventing the reservoir from sliding out of engagement with the mounting means. In the shown embodiment the pump inlet is in the form of a pointed hollow needle projecting from the pump unit prior to mounting of the reservoir, however, for the shown embodiment fluid communication between the pump unit and the reservoir is established just prior to use, this as explained in greater detail below. In an alternative embodiment (not shown) the attachment edge of the reservoir is a rounded and folded edge as shown in FIG. 1, the locking structure being adapted to engage the peripheral edges.

The pump unit further comprises a coil actuator 682 adapted to engage a piston member 683, 340 of a membrane pump (see below). In the shown embodiment the base plate 675 and the clamp member 677 are separate structures, however, these may be formed integrally with e.g. upper respectively lower housing portions.

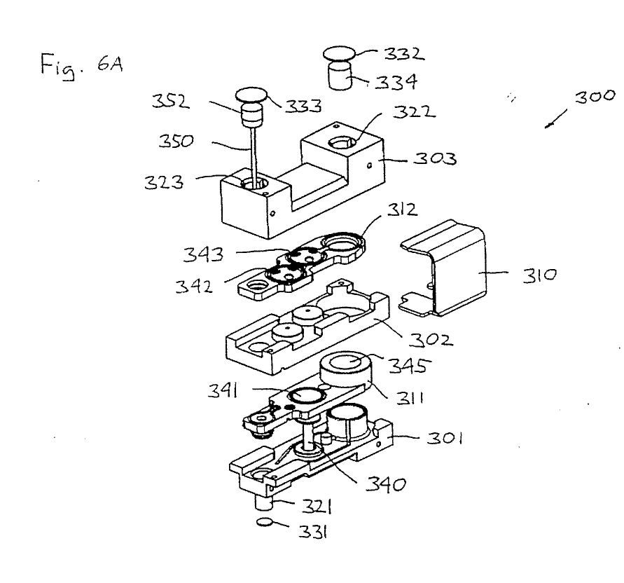

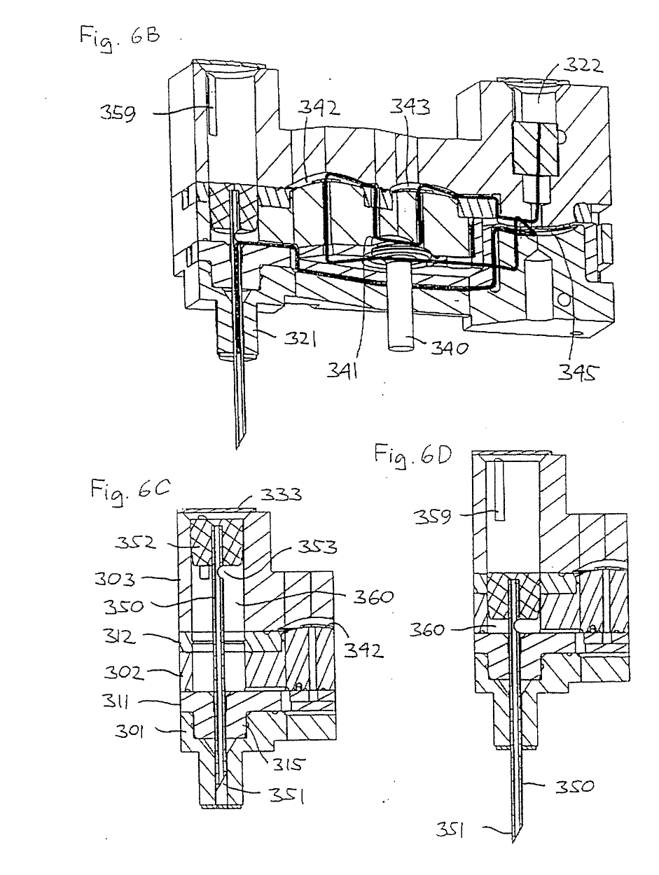

In FIG. 6A an exploded view of a pump assembly 300 is shown, the pump assembly (in the following also referred to as a pump) being suitable for use with the reservoir unit of FIG. 4. The pump is a membrane pump comprising a piston-actuated pump membrane with flow-controlled inlet- and outlet-valves. The pump has a general layered construction comprising first, second and third members 301, 302, 303 between which are interposed first and second membrane layers 311, 312, whereby a pump chamber 341 is formed by the first and second members in combination with the first membrane layer, a safety valve 345 is formed by the first and third members in combination with the first membrane layer, and inlet and outlet valves 342, 343 are formed by the second and third members in combination with the second membrane layer (see FIG. 6B). The layers are held in a stacked arrangement by an outer clamp 310. The pump further comprises an inlet 321 and an outlet 322 as well as a connection opening 323 which are all three covered by respective membranes 331, 332, 333 sealing the interior of the pump in an initial sterile state. The membranes are penetrable or breakable (e.g. made from paper) by a needle or other member introduced through a given seal. The outlet further comprises a self-sealing, needle-penetrable septa 334 (e.g. of a rubber-like material) allowing the pump to be connected to an outlet needle. As shown in FIG. 6B a fluid path (indicated by the dark line) is formed between the inlet 321 (see below) and the inlet valve 342 via the primary side of the safety valve 345, between the inlet valve, pump chamber 345 and the outlet valve 343, and between the outlet valve and the outlet 322 via the secondary side of the safety valve, the fluid paths being formed in or between the different layers. The pump also comprises a piston 340 for actuating the pump membrane, the piston being driven by an external driving assembly (not shown).

The pump further comprises a fluid connector in the form of hollow connection needle 350 slidably positioned in a needle chamber 360 arranged behind the connection opening, see FIG. 6C. The needle chamber is formed through the layers of the pump and comprises an internal sealing septum 315 through which the needle is slidably arranged, the septum being formed by the first membrane layer. The needle comprises a pointed distal end 351, a proximal end on which is arranged a needle piston 352 and a proximal side opening 353 in flow communication with the distal end, the needle and the piston being slidably arranged relative to the internal septum and the chamber. As can be appreciated form FIG. 6C the needle piston in its initial position is bypassed by one or more radially placed keyways 359. These are provided in order to allow steam sterilisation and to vent the air otherwise trapped when the fluid connector is moved forward in the needle chamber.

The above-described pump assembly may be provided in a drug delivery device of the type shown in FIGS. 4 and 5. In a situation of use where the reservoir unit is attached to a needle unit 510 the proximal end of the infusion needle 512 is introduced through the outlet seal and septum 334 of the pump, and the actuator 525 (see FIG. 4) is introduced through the connection membrane 333. By this action the connection needle is pushed from its initial position as shown in FIG. 6C to a actuated position as shown in FIG. 6D in which the distal end is moved through the inlet membrane 331 and further through the needle-penetrable septum of a nearby located reservoir, this establishing a flow path between the reservoir and the inlet valve via the proximal opening 353 in the needle. In this position a seal is formed between the needle piston and the needle chamber.

As appears, when the two units are disconnected, the proximal end of the infusion needle 512 is withdrawn from the pump outlet whereas the connection needle permanently provides fluid communication between the pump and the reservoir.

### Example

A reservoir containing 3 ml of insulin was manufactured from two foil members of a three-layered laminate (not including any tie-layer) comprising an intermediate layer of PCTFE co-extruded with epoxy modified polyethylene imine (a tie-layer) and an inner layer of PE, and with an outer layer of PE laminated on the PCTFE layer, the insulin being filled into the reservoir before it was completely sealed. A cavity-forming mould was arranged around an edge portion of the reservoir and the cavity was injected with 1-component moisture curing silicone (MED-1137 from Micro Joining KB).

The adhesives which have been found suitable as seal material have been 1-component silicone, 1-component acryl-emulsion, 1-component polyurethane, 2-component silicone, and 1-component MS polymer. The 1-component silicones are either moisture curing or UV curing. The rest of the mentioned 1-component adhesives are moisture curing and the 2-component silicone requires some post curing above room temperature. An example of a suited 1-component moisture curing silicone is the above-mentioned MED-1137 from Micro Joining KB. Moreover, Sikaflex-11FC from Sika Denmark A/S is an example of a 1-component polyurethane. Also the following materials are considered suitable as a liquid seal material: 5248 (UV curing silicone) from Loctite, Sikacryl-WT (1-component acryl-emulsion) from Sika Denmark A/S, Slkaflex-221 (1-component polyurethane) from Sika Denmark A/S, Sikasil-MP (1-component silicone) from Sika Denmark A/S, Sikaflex-11FC (1-component polyurethane) from Sika Denmark A/S, Elastosil LR 3071/40 (2-component silicone) from Medical Rubber, omniVISC 1002 (1-component silicone) from Hoejstrup Industrilim, Denmark, Liquisole (1-component polyurethane) from Casco A/S, Denmark, and Flexon HT20 (1-component MS polymer) from Diatom Vaerktoej A/S, Denmark. To improve adhesion of the adhesive to the foil material, corona treatment or other surface treatment of the foil can be used.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A reservoir (1, 560, 660) containing a fluid, comprising:
a first wall portion formed from a needle-penetrable material, the first wall portion comprising an outer seal surface,
a seal member (10, 662) formed from a needle-penetrable self-sealing material,
wherein the seal member has been applied on the seal surface in a liquid state.

2. A reservoir as defined in claim 1, in combination with a structure (676, 678, 562, 31) forming a cavity in the vicinity of the seal surface, the seal member being arranged at least partially within the cavity.

3. A reservoir as defined in claim 2, wherein the cavity-forming structure (31) is formed integrally with the reservoir.

4. A reservoir as defined in claim 2, wherein the cavity-forming structure (562) is attached to the reservoir.

5. A reservoir as defined in claim 1, in combination with a mounting structure (562), a gap being formed between the mounting structure and the seal surface, the seal member being arranged at least partially in the gap bridging a portion of the mounting structure and the seal surface.

6. A reservoir as defined in claim 1, wherein the seal member is formed from a cured material.

7. A reservoir as defined in claim 1, wherein the seal member is formed from a liquid material.

8. A reservoir as defined in claim 1, wherein the first wall portion is made from a single layer of material.

9. A reservoir as defined in claim 1, wherein the reservoir generally is formed from a single material or single type of laminate (21, 22, 23).

10. A reservoir as defined in claim 1, wherein the reservoir has an outer surface generally formed from a first material (21) and an inner surface generally formed from a second material (22).

11. A reservoir as defined in claim 1,
comprising at least first and second flexible foil members (4, 5) sealed together to form at least one enclosed cavity for containing the fluid.

12. A reservoir as defined in claim 11, wherein the foil members are composite laminates of continuous layers including an outer layer (21) and an inner layer (22).

13. A reservoir as defined in claim 12, wherein the foil members comprise an intermediate layer (23), the inner layer being formed from a weldable material, the foil members being sealed together at least partially by welding.

14. A reservoir as defined in claim 1, wherein at least a portion of the reservoir is transparent or translucent such that a contained fluid can be viewed therethrough.

15. A reservoir as defined in claim 1, wherein the seal surface is formed from a material selected from a first group of materials comprising PE, PP, OPP, PET, OPET, PA and OPA, and the seal member is formed from a material selected from a second group of materials comprising curable elastomers.

16. A reservoir device (500), comprising:
a reservoir (560) as defined in claim 15, wherein the seal member serves as a reservoir outlet,
a fluid communication structure (350) having an inlet and an outlet, the inlet being adapted to be arranged in fluid communication with the reservoir outlet (562),
wherein the fluid communication structure and the reservoir are moveable relative to each from an initial position in which there is no fluid communication therebetween and a connected position in which the fluid communication structure inlet is arranged in fluid communication with the reservoir through the reservoir outlet.

17. A reservoir device as defined in claim 16, wherein the inlet of the fluid communication structure is in the form of a pointed portion (351).

18. A reservoir device as defined in claim 17, further comprising a delivery assembly (570, 670) adapted to expel a fluid contained in the reservoir through the outlet of the reservoir.

19. A reservoir device as defined in claim 17, further comprising a delivery assembly (570, 670) having an inlet (321) associated with the fluid communication structure and an outlet (322), the inlet being adapted to be arranged in fluid communication with the reservoir, the delivery assembly being capable of expelling a fluid contained in the reservoir through the outlet of the delivery assembly.

20. A reservoir device as defined in claim 19, further comprising:
a transcutaneous access device (512) adapted to be arranged through the skin of a subject,
a mounting surface (532) adapted for application to the skin of the subject, wherein
the reservoir comprises a liquid drug, and
wherein the delivery assembly, in a situation of use, is adapted for expelling the drug out of the reservoir and through the skin of the subject via an outlet end of the transcutaneous access device.

21. A method for manufacturing a reservoir (1, 560, 660) adapted to contain a fluid, comprising the steps of:
providing a reservoir comprising a first wall portion formed from a needle-penetrable material, the first wall portion comprising an outer seal surface, and
applying a liquid material to the seal surface, the liquid material being adapted to form a needle-penetrable self-sealing seal member (10, 662).

22. A method as defined in claim 21, wherein the reservoir is provided in combination with a structure (676, 678) forming a cavity, the cavity being arranged to receive at least a portion of the applied liquid material.

23. A method as defined in claim 21, further comprising the step of curing or allowing the liquid material to cure, thereby forming the needle-penetrable self-sealing seal member.

24. A method as defined in claim 21, wherein the reservoir is sealed and contains an amount of a fluid prior to the application of the liquid material.
